(19) United States
(12) Patent Application Publication
(10) Pub. No.: US 2022/0362427 A1
(43) Pub. Date: Nov. 17, 2022

(54) BATTERY POWERED SELF-ADJUSTING SANITIZER/DISINFECTANT SPRAYER

(71) Applicant: GOJO Industries, Inc., Akron, OH (US)

(72) Inventors: Mark W. Moore, Seven Hills, OH (US); Nick E. Ciavarella, Seven Hills, OH (US); Mark S. Kacik, Avon Lake, OH (US); Jackson W. Wegelin, Stow, OH (US); John J. McNulty, Broadview Heights, OH (US)

(21) Appl. No.: 17/734,459

(22) Filed: May 2, 2022

Related U.S. Application Data
(60) Provisional application No. 63/187,000, filed on May 11, 2021.

(51) Int. Cl. A61L 2/24; A61L 2/22

(52) U.S. Cl. CPC A61L 2/24; A61L 2/22; A61L 2202/14; A61L 2202/15; A61L 2202/16

(57) ABSTRACT

Exemplary embodiments of self-adjusting sanitizer/disinfectant sprayers are disclosed herein. An exemplary self-adjusting sanitizer/disinfectant sprayer includes a tank for holding sanitizer or disinfectant, one or more batteries, a motor, a motor controller, and a pump. The pump includes a pump inlet in fluid communication with an interior of the tank and a pump outlet. A processor, a dispensing wand and a flow sensor are also included. The flow sensor and motor controller are in circuit communication with the processor. The processor provides input to the motor controller for controlling the speed of the motor that drives the pump as a function of a signal indicative of the flow rate received from the flow sensor.

### RELATED APPLICATION

This application claims priority to and any benefit of U.S. Provisional Application No. 63/187,000, filed May 11, 2021, the content of which is incorporated herein by reference in its entirety.

### TECHNICAL FIELD

The present invention relates generally to battery powered liquid sprayers and more particularly to battery powered self-adjusting sprayers having feedback control for adjusting or maintaining desired spray characteristics.

### BACKGROUND OF THE INVENTION

Battery powered fluid sprayers are convenient because a user does not need to repeatedly manually operate a pump to pump the fluid or pressurize the fluid tank. Battery powered fluid sprayers typically provide inconsistent spray characteristics over the life or charge of the battery. Inconstant spray characteristics often result in varying amounts of disinfectant being applied to a surface at any given time. Accordingly, even experienced users often apply too much fluid to a surface or not enough fluid to the surface, which results in over-wetting of the surface or inefficacious amounts of sanitizer/disinfectant being applied to the surface. Over-wetting may result in excess consumption of the sanitizer/disinfectant and may create slipping hazards. Applying inefficacious amounts of sanitizer/disinfectant to a surface may result in not sanitizing or disinfecting the surface. Accordingly, there is a need for a battery powered fluid sprayer that utilizes feedback sensor to control and/or maintain selected sprayer spray characteristics.

### SUMMARY

Exemplary embodiments of self-adjusting sanitizer/disinfectant sprayers are disclosed herein. An exemplary self-adjusting sanitizer/disinfectant sprayer includes a tank for holding sanitizer or disinfectant, one or more batteries, a motor, a motor controller, and a pump. The pump includes a pump inlet in fluid communication with an interior of the tank and a pump outlet. A processor, a dispensing wand and a flow sensor are also included. The flow sensor and motor controller are in circuit communication with the processor. The processor provides input to the motor controller for controlling the speed of the motor that drives the pump as a function of one or more signals indicative of the flow rate received from the flow sensor.

Another exemplary self-adjusting sanitizer/disinfectant sprayer includes a tank for holding sanitizer or disinfectant, one or more batteries, a motor, a motor controller, a pump, a processor, a dispensing wand, and a sensor for sensing a parameter indicative of a flow rate. The self-adjusting sprayer further includes circuitry for monitoring the voltage of the battery and circuitry for preventing operation of the sanitizer/disinfectant sprayer if the voltage of the battery falls below a selected voltage or a selected flow rate is below a set threshold for greater than a selected time period.

Another exemplary self-adjusting sprayer includes a tank for holding sanitizer or disinfectant, one or more batteries, a motor, a motor controller, a pump, a processor, a dispensing wand, and one or more sensors selected from the group of an accelerometer sensor, a gyroscope sensor, a magnetometer sensor a velocimeter sensor, a time of flight sensor, an imaging sensor and a distance sensor. The processor utilizes data from at least one of the sensors to adjust a flow of fluid flowing out of the dispensing wand.

Another exemplary self-adjusting sanitizer/disinfectant sprayer includes a tank for holding sanitizer or disinfectant, one or more batteries, a motor, a motor controller, a pump, a processor, a dispensing wand and a distance sensor. The distance sensor is in circuit communication with the processor, which is also in circuit communication with the motor controller. The processor provides input to the motor controller for controlling the speed of the motor that drives the pump as a function of a signal indicative of the distance received from the distance sensor to a target.

Another exemplary self-adjusting sprayer includes a tank for holding sanitizer or disinfectant, one or more batteries, a motor, a motor controller, a pump, a processor, memory, a dispensing wand, one or more feedback sensors, and logic stored on the memory. The logic stored on the memory causes the processor to change one or more fluid dispensing properties as a function of data received from the one or more feedback sensors.

### BRIEF DESCRIPTION OF THE DRAWINGS

These and other features and advantages of the present invention will become better understood with regard to the following description and accompanying drawings in which:

FIG. 1 is simplified schematic view of an exemplary embodiment of a self-adjusting sanitizer/disinfectant sprayer having feedback control;

FIG. 2 is simplified schematic diagram of another exemplary embodiment of a self-adjusting sanitizer/disinfectant sprayer having feedback control;

FIG. 3 is simplified schematic diagram of yet another exemplary embodiment of a self-adjusting sanitizer/disinfectant sprayer having feedback control;

FIG. 4 is a logic diagram for an exemplary embodiment for a self-adjusting sanitizer/disinfectant sprayer having feedback control;

FIG. 5 is a logic diagram for another exemplary embodiment for a self-adjusting sanitizer/disinfectant sprayer having feedback control;

FIG. 6 is a logic diagram for another exemplary embodiment for a self-adjusting sanitizer/disinfectant sprayer having feedback control;

FIG. 7 is simplified schematic diagram of another exemplary embodiment of a self-adjusting sanitizer/disinfectant sprayer having feedback control;

FIG. 8 is simplified schematic view of another exemplary embodiment of a sanitizer/disinfectant sprayer having feedback control that may be self-adjusting and/or may provide one or more indications for a user to make adjustments;

FIG. 9 is a logic diagram for an exemplary embodiment of a sanitizer/disinfectant sprayer having feedback control that may be self-adjusting and/or may provide one or more indications for a user to make adjustments;

FIG. 10 is simplified schematic view of another exemplary embodiment of a sanitizer/disinfectant sprayer having feedback control that may be self-adjusting and/or may provide one or more indications for a user to make adjustments;

FIG. 11 is simplified schematic view of another exemplary embodiment of a self-adjusting sanitizer/disinfectant sprayer having feedback control;

FIG. 12 is a logic diagram of another exemplary embodiment of a self-adjusting sanitizer/disinfectant sprayer having feedback control; and

FIG. 13 is a logic diagram of yet another exemplary embodiment of a self-adjusting sanitizer/disinfectant sprayer having feedback control.

### DETAILED DESCRIPTION

Exemplary embodiments for sanitizer/disinfectant sprayers having feedback control are disclosed herein.

The following includes definitions of exemplary terms used throughout the disclosure. Both singular and plural forms of all terms fall within each meaning. Except where noted otherwise, capitalized and non-capitalized forms of all terms fall within each meaning:

“Circuit communication” as used herein indicates a communicative relationship between devices. Direct electrical, electromagnetic and optical connections and indirect electrical, electromagnetic and optical connections are examples of circuit communication. Two devices are in circuit communication if a signal from one is received by the other, regardless of whether the signal is modified by some other device. For example, two devices separated by one or more of the following—amplifiers, filters, transformers, optoisolators, digital or analog buffers, analog integrators, other electronic circuitry, fiber optic transceivers or satellites—are in circuit communication if a signal from one is communicated to the other, even though the signal is modified by the intermediate device(s). As another example, an electromagnetic sensor is in circuit communication with a signal if it receives electromagnetic radiation from the signal. As a final example, two devices not directly connected to each other, but both capable of interfacing with a third device, such as, for example, a CPU, are in circuit communication.

Also, as used herein, any voltages and values representing digitized voltages are considered to be equivalent for the purposes of this application, and thus the term “voltage” as used herein refers to either a signal, or a value in a processor representing a signal, or a value in a processor determined from a value representing a signal.

“Signal”, as used herein includes, but is not limited to one or more electrical signals, analog or digital signals, one or more computer instructions, a bit or bit stream, or the like.

“Logic,” synonymous with “circuit,” as used herein includes, but is not limited to hardware, firmware, software and/or combinations of each to perform a function(s) or an action(s). For example, based on a desired application or needs, logic may include a software controlled microprocessor or microcontroller, discrete logic, such as an application specific integrated circuit (ASIC) or other programmed logic device. Logic may also be fully embodied as software. The circuits identified and described herein may have many different configurations to perform the desired functions.

Values identified in the detailed description are exemplary and they are determined as needed for a particular dispenser and/or refill design. Accordingly, the inventive concepts disclosed and claimed herein are not limited to the particular values or ranges of values used to describe the embodiments disclosed herein.

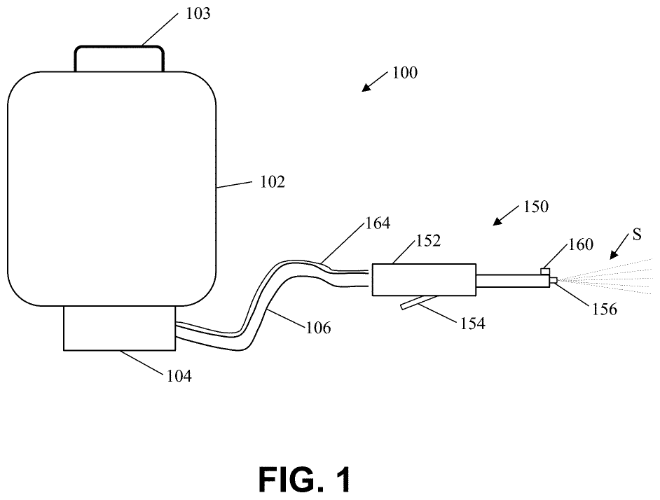

FIG. 1 is simplified schematic view of an exemplary embodiment of a self-adjusting sanitizer/disinfectant sprayer 100 having feedback control. In this exemplary embodiment, self-adjusting sanitizer/disinfectant sprayer 100 includes a tank 102 for holding sanitizer/disinfectant, a pump housing 104, a liquid feed conduit 164, a spray wand 152, outlet nozzle 156, feedback sensor 160, trigger 154 and cable 164. Tank 102 includes a carrying handle 103. Pump housing 104 includes a battery power supply (not shown), control circuitry (not shown) and a pump (not shown). Cable 164 places the trigger 154 and sensor 160 in circuit communication with the control circuitry.

In this exemplary embodiment feedback sensor 160 is a flow sensor. Flow sensor 160 may be located proximate the end of wand 150 as illustrated, or it may be located in the pump housing 104, in liquid feed conduit 106, or any other location where it is capable of determining or sensing the flow rate of the fluid flowing out of nozzle 156.

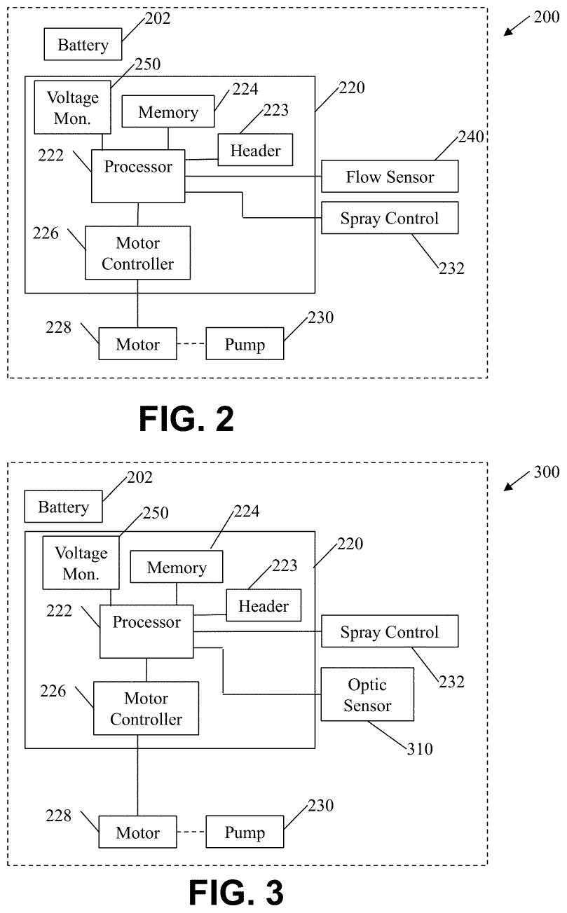

FIG. 2 is simplified schematic diagram of an exemplary embodiment of self-adjusting sanitizer/disinfectant sprayer 200 having feedback control. Self-adjusting sanitizer/disinfectant sprayer 200 includes a battery power pack 202. Battery power pack 202 provides power to control circuitry 220, flow sensor 240, motor controller 226, motor 228 and any other device that requires power. The term “battery pack” should be construed broadly to mean one or more batteries. When more than one battery are include in the battery pack, the one or more batteries may be connected in series, may be connected in parallel, or combinations thereof.

In some embodiments, battery power pack 202 or control circuitry 220 includes voltage regulation circuitry (not shown). In some embodiments, the voltage regulation circuitry is included in system circuitry 220. One or more components shown on system circuitry 220 may be mounted on a common circuit board and/or may be separately mounted and placed in circuit communication with the required other components.

Processor 222 may be any type of processor, such as, for example, a microprocessor or microcontroller, discrete logic, such as an application specific integrated circuit (ASIC), other programmed logic devices or the like. Processor 222 is in circuit communication with optional header 223. Header 223 is a circuit connection port that allows a user to connect to system circuitry 220 to program the circuitry, run diagnostics on the circuitry and/or retrieve information from the circuitry.

Processor 222 is in circuit communication with memory 224. Depending on the need, memory 224 may be any type of memory, such as, for example, Random Access Memory (RAM); Read Only Memory (ROM); programmable read-only memory (PROM), electrically programmable read-only memory (EPROM), electrically erasable programmable read-only memory (EEPROM), flash, magnetic disk or tape, optically readable mediums including CD-ROM and DVD-ROM, or the like, or combinations of different types of memory. In some embodiments, the memory 224 is separate from the processor 222, and in some embodiments, the memory 224 resides on or within processor 222.

Processor 222 is in circuit communication with motor controller 226. Motor controller 226 may be any type of circuitry used to control motor 228. Preferably, motor controller 226 utilizes pulse width modulation control to control the speed of motor 228. A detailed description of pulse width modulation control may be found in Applicants co-pending U.S. patent application Ser. No. 16/176,411, which is titled TOUCH-FREE DISPENSERS and was filed on Oct. 31, 2018; and also in Applicants U.S. Pat. Pub. No. 2017-0049276 title POWER SYSTEMS FOR DYNAMICALLY CONTROLLING A SOAP, SANITIZER OR LOTION DISPENSER DRIVE MOTOR, each of which is incorporated herein by reference in their entirety.

In this exemplary embodiment, system circuitry 220 includes optional voltage monitoring circuitry 250 which monitors the voltage of battery pack 202. As discussed in more detail below, voltage monitoring circuitry 250 may be used by processor 222 to cut-off operation of sanitizer/disinfectant sprayer 200 when the voltage output of the battery pack falls below about 3 volts, or falls below about 2.8 volts; or falls below about 2.5 volts; or falls below about 2 volts. In some embodiments, voltage monitoring circuitry 250 may be used by processor 222 to cut-off operation of sanitizer/disinfectant sprayer 200 when the voltage output of the battery pack falls below a set percentage of the full charge rating of the battery pack, such as, for example, 50% of the rated capacity, 45% of the rated capacity, 40% of the rated capacity, 35% of the rated capacity, 30% of the rated capacity, 33% of the rated capacity 25% of the rated capacity, or 20% of the rated capacity. In some embodiments, processor 222 prevents operation of the sprayer if the battery voltage is below any of the above identified ranges. The voltages or capacities are above a threshold at which the battery stops providing enough power to power the motor. In some embodiments, the threshold is selected so that the flow rate is at least about 95% of the flow rate of a fully charged battery. In some embodiments, the threshold is selected so that the flow rate is at least about 90% of the flow rate of a fully charged battery. In some embodiments, the threshold is selected so that the flow rate is at least about 85% of the flow rate of a fully charged battery. In some embodiments, the threshold is selected so that the flow rate is at least about 80% of the flow rate of a fully charged battery. In some embodiments, the threshold is selected so that the flow rate is at least about 75% of the flow rate of a fully charged battery. In some embodiments, the threshold is selected so that the flow rate is at least about 70% of the flow rate of a fully charged battery.

Processor 222 provides one or more outputs to motor controller 226, which drives motor 228. Motor 228 drives pump 230, or drives an actuator (not shown) and/or one or more gears that drives pump 230. In some embodiments, motor controller 226 is designed to provide consistent power to the motor 228 irrespective of the actual voltage of the battery pack 202. In some embodiments, pulse width modulation circuitry is used to accomplish consistent power. In some embodiments, when the charge of the battery pack is high, the voltage pulse width delivered to the motor 228 is short. As the charge of the battery pack decreases, the voltage pulse width delivered to the motor 228 is lengthened. Accordingly, the speed of the motor 228 may be controlled or maintained irrespective of the charge on the battery pack 202.

Processor 232 is in circuit communications with spray control 232. Spray control 232 initiates dispensing or spraying of sanitizer/disinfectants. Spray control 232 may be a trigger on a dispensing wand (not shown) that is used to direct sanitizer/disinfectant onto desired surfaces. In some embodiments, spay controller 232 may be a button, voice activated controller, a switch, or the like, Spray control 232 may be hard wired to system circuitry 220. In some embodiments, system circuitry 220 includes optional wireless communications circuitry (not shown) or receiving and/or transmitting signals to one or more devices. In some embodiments, the dispensing wand (not shown) includes wireless communications circuitry for transmitting and/or receiving signals. In some embodiments, a trigger (not shown) on a dispensing wand (not shown) is in wireless circuit communications with processor 222.

A flow sensor 240 is also in circuit communications with processor 222. Flow sensor 240 is used to monitor the flow of sanitizer/disinfectant that is flowing out of the sprayer wand (not shown). Flow sensor 240 may be any sensor that senses flow of fluid flowing through and/or out of the system. In some embodiments, flow sensor 240 is an in-line flow sensor, i.e. flow sensor 240 directly monitors the flow through a fluid conduit and/or portion of the wand. Exemplary flow sensors include, differential pressure flow meters, positive displacement flow meters, velocity flow meters, mass flow meter, turbine meters, ultrasonic meters, and the like. In addition, flow sensor 240 may be an optical flow sensor. The optical flow sensor may use an optical sensor to detect droplet size, droplet velocity or the like that is indicative of the fluid flow rate.

In some embodiments, it is important to control the flow rate of the fluid that is flowing out of the sprayer wand. The flow rate has direct impact on, for example, droplet size, spray distance, volumes of fluid sprayed per unit of time, size of a spray patch on a surface, evenness of spray and the like. During operation, processor 222 receives a signal indicative of the flow rate from flow sensor 240. If the flow rate is below a selected threshold, the processor 222 provides instructions for the motor controller 226 to increase the speed of the motor 228. If the flow rate is above a selected threshold, the processor 222 provides instructions for the motor controller 226 to decrease the speed of the motor 228.

In some embodiments, flow sensor 240 is optional. In those embodiments, processor 222 cuts off power to the motor 228 if the voltage monitoring circuitry determines that the voltage or power of the battery 202 is below a selected power threshold or voltage threshold, such as, for example, the % rated capacities identified above, or the voltages identified herein. The thresholds are selected to be above a threshold where the battery stops providing enough power to turn the motor.

FIG. 3 is simplified schematic diagram of an exemplary embodiment of a self-adjusting sanitizer/disinfectant sprayer 300 having feedback control. Sanitizer/disinfectant sprayer 300 is similar to self-adjusting sanitizer/disinfectant sprayer 200 and components with the same reference numbers are not redescribed with respect to this exemplary embodiment. Flow sensor 240 has been replaced by optic sensor 310. Optic sensor 310 captures spray pattern images. The spray pattern images may be compared to spray pattern images stored in the memory 224. Each stored spray pattern image may correlate to a selected motor speed. In some embodiments, a selected spray pattern image may be chosen or preset for the sprayer. If the spray pattern detected by optic sensor 310 is different from the desired spray pattern, processor 222 increases or decreases the speed of the motor to arrive at the desired spray pattern. In some embodiments, the processor increases the speed of the motor and determines if the detected spray pattern image is getting closer to the selected spray pattern or further away from the selected spray pattern. If the detected spray pattern is getting closer to the selected spray pattern, the processor 222 continues to increase the speed until the detected spray pattern is close to the selected spray pattern. If the detected spray pattern is getting further away from the selected spray pattern, processor 222 decreases the speed of the motor until the detected spray pattern is close to the selected spray pattern

The exemplary methodologies described herein contain a number of blocks or steps. Additional blocks or steps may be added to these exemplary embodiments. In addition, some blocks or steps may be removed from the exemplary methodologies. Further, blocks or steps from exemplary methodologies disclosed herein may be included in other methodologies or logic diagrams disclosed herein. In addition, unless expressly stated otherwise, the order in which the steps are performed is not critical and may be changed.

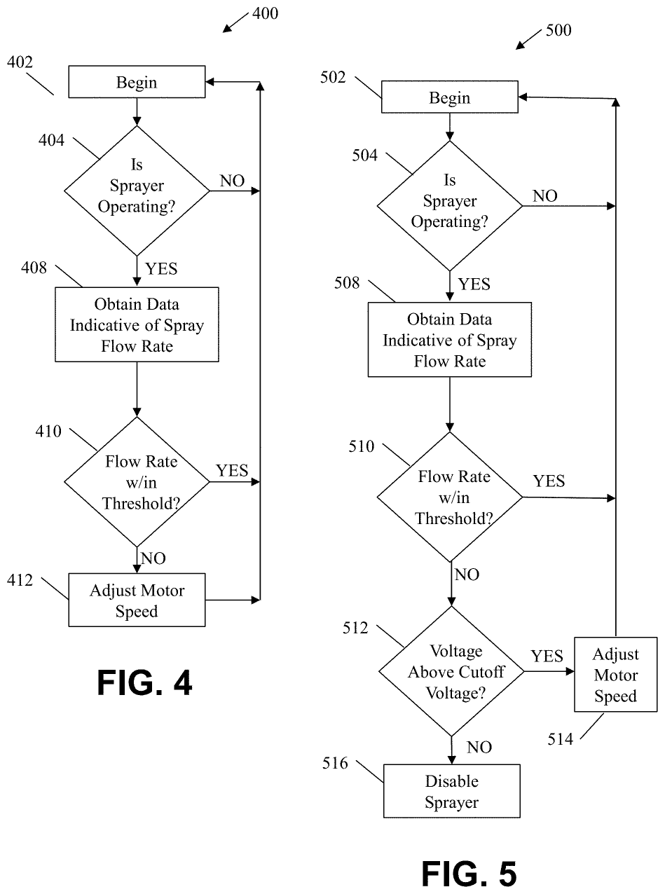

FIG. 4 is a logic diagram or methodology for controlling an exemplary embodiment of a self-adjusting sanitizer/disinfectant sprayer having feedback control. The exemplary logic diagram begins at block 402. At block 404 a determination is made as to whether a request for the sprayer to spraying fluid has been initiated. If there is no request, the methodology loops back to block 402. If a request to spray fluid has been initiated, the methodology flows to block 408 to obtain data indicative of the spray flow rate. A determination is made at block 410 as to whether the flow rate is within a selected threshold. Exemplary thresholds may be, for example, within 0.05%, within 0.1%, within 0.5%, within 1%, within 2%, within 3%, within 4%, within 5%, or within 10%. In some exemplary embodiments, flow rate is estimated based on motor speed, and the threshold may be applied to the speed of the motor. If the flow rate is within the threshold, the exemplary methodology loops back to block 402. If the flow rate is outside of the threshold, the exemplary methodology flows to block 412 wherein the speed of the motor is adjusted to bring the flow rate back to being within the selected threshold. Preferably this methodology is continuous throughout the spraying operation. In some embedment's, the methodology is used periodically, such as, for example, ever 10 seconds, every 20 seconds, every minute of operation. Preferably, the methodology begins each time the sprayer is activated.

FIG. 5 is another logic diagram or methodology 500 for controlling an exemplary embodiment of sanitizer/disinfectant sprayer having feedback control. The exemplary logic diagram begins at block 502. At block 504 a determination is made as to whether the sprayer is spraying fluid. If the sprayer is not spraying fluid, the methodology loops back to block 502. If the sprayer is spraying fluid, the methodology flows to block 508 to obtain data indicative of the spray flow rate. A determination is made at block 510 as to whether the flow rate is within a selected threshold. Exemplary thresholds have been described above. If the flow rate is within the threshold, the exemplary methodology loops back to block 502. If the flow rate is outside of the threshold, the exemplary methodology flows to block 512 where a determination is made as to whether the voltage of the battery pack is above a cut-off voltage. If the voltage is above the cut-off voltage, the exemplary methodology flows to block 514 and the speed of the motor is adjusted. If it is determined that the battery pack voltage is not above the cut-off voltage, the exemplary methodology flows to block 516 and the sprayer is disabled or prevented from operating.

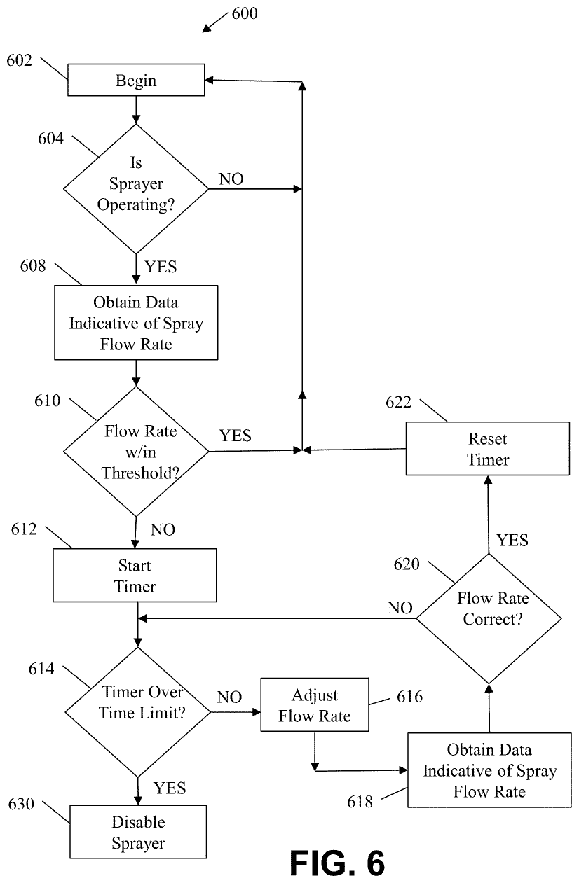

FIG. 6 is another logic diagram or methodology for controlling an exemplary embodiment of sanitizer/disinfectant sprayer having feedback control. The exemplary logic diagram begins at block 602. At block 604 a determination is made as to whether the sprayer is spraying fluid. If the sprayer is not spraying fluid, the methodology loops back to block 602. If the sprayer is spraying fluid, the methodology flows to block 608 to obtain data indicative of the spray flow rate. A determination is made at block 610 as to whether the flow rate is within a selected threshold. Exemplary thresholds have been described above. If the flow rate is within the threshold, the exemplary methodology loops back to block 602. If the flow rate is outside of the threshold, the exemplary methodology flows to block 612 and a timer is started. The exemplary methodology flows to block 614 where a determination is made as to whether the timer is over the set time limit. If the timer is not over the set time limit, the methodology flows to block 616 wherein one or more parameters are changed to adjust the flow rate. At block 618 data indicative of the flow rate is obtained and at block 620 a determination is made as to whether to flow rate is consistent with the adjusted flow rate. If the flow rate is at the set point, the timer is reset at block 622 and the methodology flows to block 602. If the flow rate is not up to the adjusted flow rate, the methodology loops back to block 614 where a determination is made as to whether the timer has timed out. If the timer has timed out, the sprayer is disabled or prevented from operating at block 630.

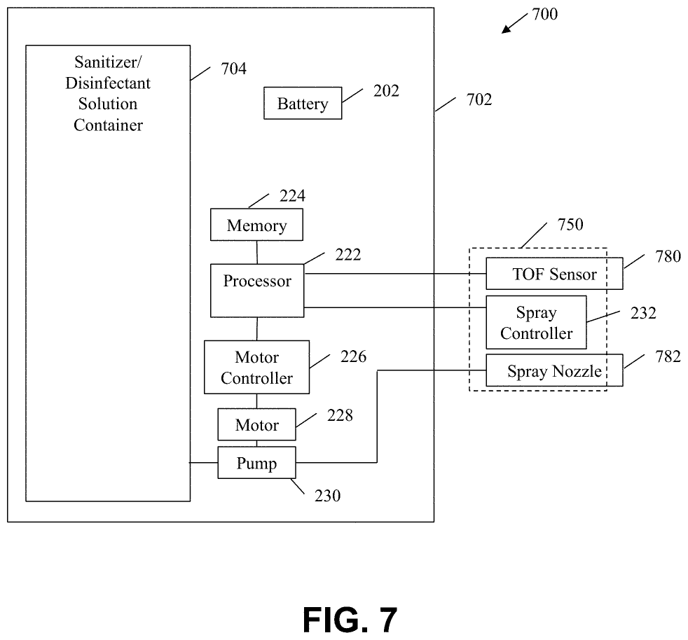

FIG. 7 is simplified schematic diagram of another exemplary embodiment of a self-adjusting sanitizer/disinfectant sprayer having feedback control. Sanitizer/disinfectant sprayer 700 includes a battery pack 202, a processor 222, memory 224, a motor controller 226, a motor 228, a pump 230 and a spray controller 232. These components are similar to those described with respect to the embodiment shown in FIG. 2 and described in detail above, and accordingly, are not redescribed in detail herein. Sanitizer/disinfectant sprayer 700 includes a housing 702, a container 704 for holding sanitizer or disinfectant. The container 704 is in fluid communication with the pump 230, which is in fluid communication with the spray nozzle 782. One or more valves, such as, for example, one-way valves may be incorporated into the flow path. In addition, Sanitizer/disinfectant sprayer 700 includes a time of flight (“TOF”) sensor 780, or a distance sensor, in circuit communications with processor 222. The TOF sensor 780 is located in the wand 750. The TOF sensor 780 measures the distance to a target in front of the wand 780 and provides a signal indicative of the distance to the processor 222. Processor 222 utilizes the signal to control the speed of the motor and/or the flow rate of the fluid to the wand 750.

In some embodiments, the Sanitizer/disinfectant sprayer 700 is configured to dispense fluid at a first flow rate that is set for a targeted distance. The TOF sensor 780 determines the distance to an object that is in front of the wand 750. The object, may be, for example, a wall, a desk, a counter, a device, or the like. If the TOF sensor 780 detects a distance to the object that is less than the targeted distance, processor 222 reduces the flow rate of fluid to the wand 750. If the TOF sensor 780 detects a distance to the object that is greater than the targeted distance, processor 222 increases the flow rate of fluid to the wand 750. In some embodiments, if the TOF sensor 780 detects that the object is outside a selected range, processor 222 prevents sanitizer/disinfectant sprayer 700 from spraying fluid.

In some embodiments, if the TOF sensor 780 senses a rapid change in distance, processor 222 stops sanitizer/disinfectant sprayer 700 from spraying fluid. This feature is useful in preventing overspray which is a waste of sanitizer/disinfectant and also may lead to slipping hazards. For example, if a janitor is sanitizing or disinfecting desks in a class room, when the wand 750 gets to the end of the desk, the sanitizer/disinfectant sprayer 700 shuts off and stops dispensing sanitizer or disinfectant as soon as the TOF sensor 780 detects a rapid change in distance, i.e. the wand 750 passed over the end of the desk.

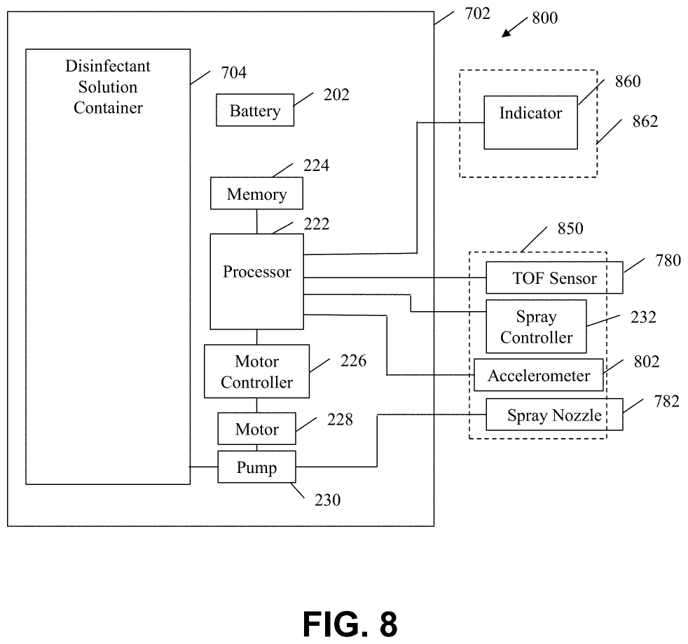

FIG. 8 is simplified schematic diagram of another exemplary embodiment of sanitizer/disinfectant sprayer having feedback control. Sanitizer/disinfectant sprayer 800 is similar to sanitizer/disinfectant sprayer 700 and like components are not redescribed herein. Sanitizer/disinfectant sprayer 800 includes an accelerometer 802 located in wand 850. Accelerometer is in circuit communications with processor 222. Accelerometer 802 provides a feedback signal to processor 222. Processor 222 may use the feedback signal to increase or decrease the flow rate of the sanitizer or disinfectant solution. For example, if the accelerometer 802 signal indicates a rapid acceleration, processor 222 increases the flow rate. If the accelerometer 802 signal indicates a rapid deceleration, processor 222 may decrease the flow rate. The accelerometer 802 may be useful for applications where the operator is using sweeping motions to disinfect a surface, such as, for example, a counter top or table.

In addition, sanitizer/disinfectant sprayer 800 includes one or more optional indicators 860. One or more optional indicators 860 may provide a visual, audible, and/or haptic signal to the operator of the sanitizer/disinfectant sprayer 800. Thus in this exemplary embodiment, the sprayer 800 may be a “self-adjusting” by directing an operator to make adjustments in the operator's use of the sprayer. Such indicators, may include, for example, one or more lights, such as a green light and a red light. The green light may indicate that the operator is moving within a desired speed range, while the red light may indicate operation outside of the desired speed range. In some embodiments, a first light (e.g. yellow light) means that the operator has the wand 850 too close to the surface, a second light (e.g. an orange light) means that the operator is too far away from the surface, and a third light (e.g. a blue light) means the operator has the wand 850 a correct distance from the surface.

Audible indicators may be, for example, a voice synthesizer that provides audible messaging to the operator to change an application characteristic, such as, for example, speed, consistency, motion, and the like. Similarly the haptic signal may provide, for example, a vibratory sensation in the wand if the operator is operating outside of one or more characteristics. In some embodiments, two short vibrations may mean that the operator is moving too fast and one long vibration may mean the operator is moving to slow.

The one or more indicators 860 may be training indicators that are used to teach or remind operators of the proper use of the sanitizer/disinfectant sprayer 800.

The one or more indicators 860 may be in a housing 862. The housing 862 may be attached to the sprayer housing 702, the wand 850, or the operator. In some embodiments, housing 862 is in the form of a wearable device, such as, for example, a badge, a smart phone, or the like.

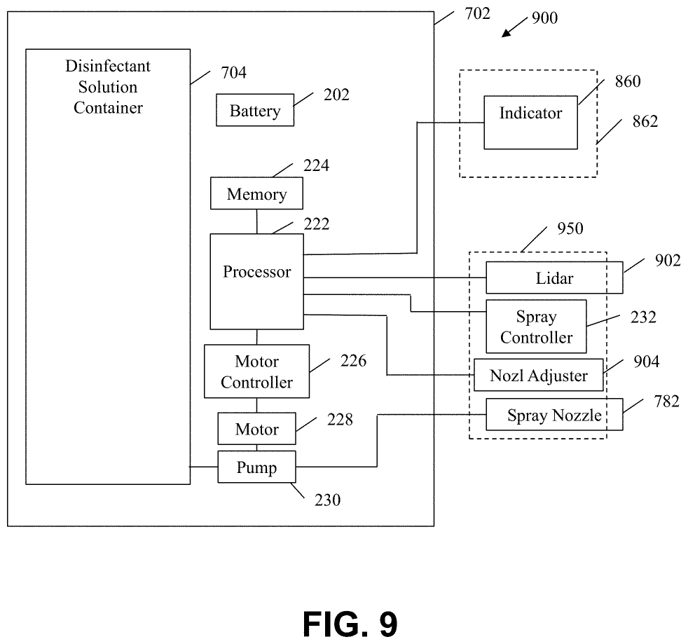

FIG. 9 is simplified schematic diagram of another exemplary embodiment of a self-adjusting sanitizer/disinfectant sprayer having feedback control. Sanitizer/disinfectant sprayer 900 is similar to sanitizer/disinfectant sprayer 800 and like components are not redescribed herein. Sanitizer/disinfectant sprayer 900 includes a LiDar sensor 902 in circuit communications with processor 222. In addition, sanitizer/disinfectant sprayer 900 includes an optional nozzle adjuster 904 in circuit communication with processor 222.

LiDar sensor 902 utilizes pulsed laser signals to generate a 3-D image of an area in its field of view, such as, for example, an object that is being sanitized and or disinfected. Accordingly, processor 222 may be configured to increase or decrease the flow rate of sanitizer or disinfectant that is being applied to the surface of the object. The increase or decrease may be a function of distance to points on the surface of the object, speed at which the wand is moving, or the like.

Optional nozzle adjuster 904 may be used by processor 222 to adjust the droplet size being applied to the object. In some examples, if an object surface is close to the wand 950, processor uses nozzle adjuster to adjust the nozzle to deliver finer droplet sizes on the surface. As the wand 950 is moved away from the surface, processor 222 uses nozzle adjuster to increase the droplet size that is being dispensed.

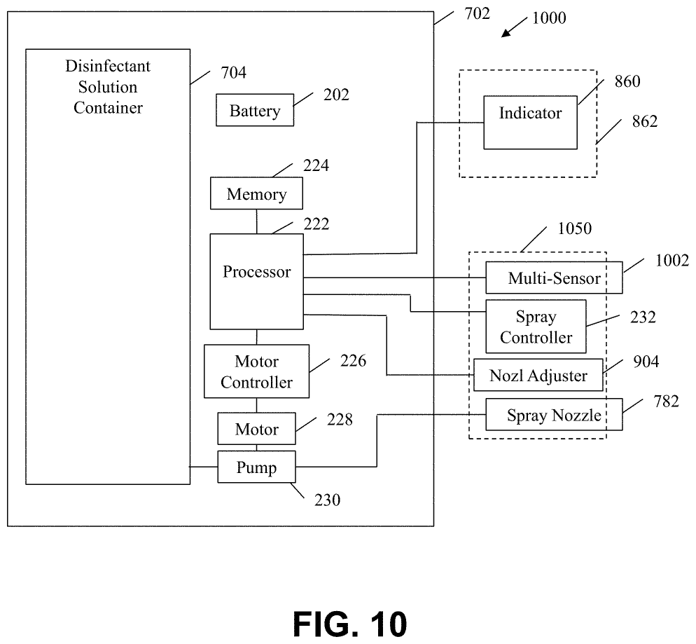

FIG. 10 is simplified schematic diagram of another exemplary embodiment of a self-adjusting sanitizer/disinfectant sprayer having feedback control. Sanitizer/disinfectant sprayer 1000 is similar to sanitizer/disinfectant sprayer 900 and like components are not redescribed herein. Sanitizer/disinfectant sprayer 1000 includes a multi-sensor 1002 in circuit communications with processor 222. Multi-sensor 1002 may be one or more sensors. Multi-senor 1002 includes one or more of an accelerometer, a gyroscope, a magnetometer, a velocimeter, a time of flight sensor, a distance sensor or the like. In this exemplary embodiment, processor 222 may precisely control the volume of fluid being dispensed on the surface of an object and may precisely stop dispensation of fluid as the wand 1050 travels past the ends of the objects. Very precise control of the fluid is possible because processor 222 can determine multiple variables, including two or more of acceleration, orientation, velocity, and distance to the object that is being sanitized or disinfected.

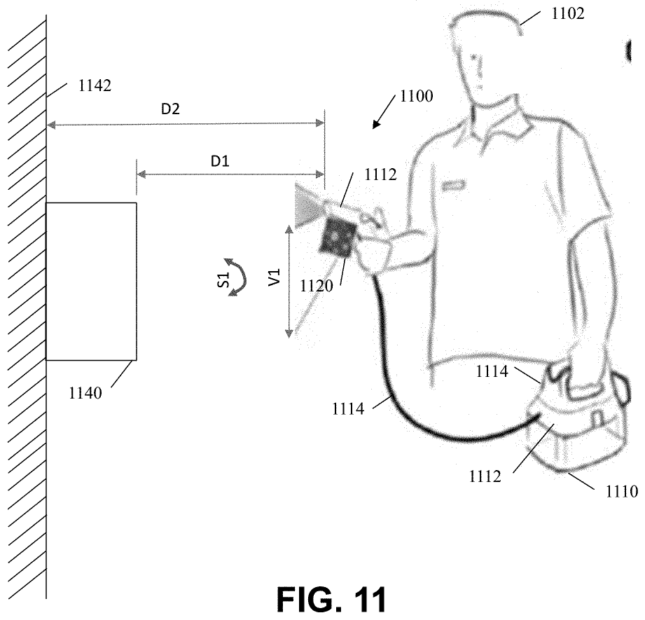

FIG. 11 is simplified schematic view of another exemplary embodiment of sanitizer/disinfectant sprayer 1100 having feedback control. Sanitizer/disinfectant sprayer 1100 includes a tank 1110 for holding sanitizer/disinfectant, a pump house 1112, a handle 1114, disinfectant/sanitizer fluid conduit 1114, a spray wand 1112 and a feedback sensor 1120. These components may be similar to like components described above. Feedback sensor 1120 may be any of the sensors described above. In some embodiments, feedback sensor 1120 includes a distance sensor. In some embodiments, feedback sensor includes one or more of an accelerometer, a gyroscope, a magnetometer a velocimeter, or the like. In this exemplary embodiment, feedback sensor 1120 may detect a distance D1 to an object 1140. A processor (not shown) located in the pump house 1112 adjusts the flow rate of sanitizer/disinfectant flowing out of the dispensing wand 1112. The flow rate may be set as a function of the distance D1. In this exemplary embodiment, as dispensing wand 1112 moves past the edge of object 1140, feedback sensor 1120 detects an abrupt increase in distance to distance D2 and the processor (not shown) stops fluid flow, which prevents overspray. If the user moves the dispensing wand 1112 back down, distance D1 is detected and the processor starts fluid flow (provided that the trigger (not shown) is pressed. In some embodiments, feedback sensor 1120 can sense a sweeping motion S 1. In such an embodiment, the processor (not shown) may increase and decrease flow as a function of the location of the dispensing wand 1112 in the sweep S 1. In some embodiments, feedback sensor 1120 detects acceleration and deceleration and increases and decreases flow accordingly.

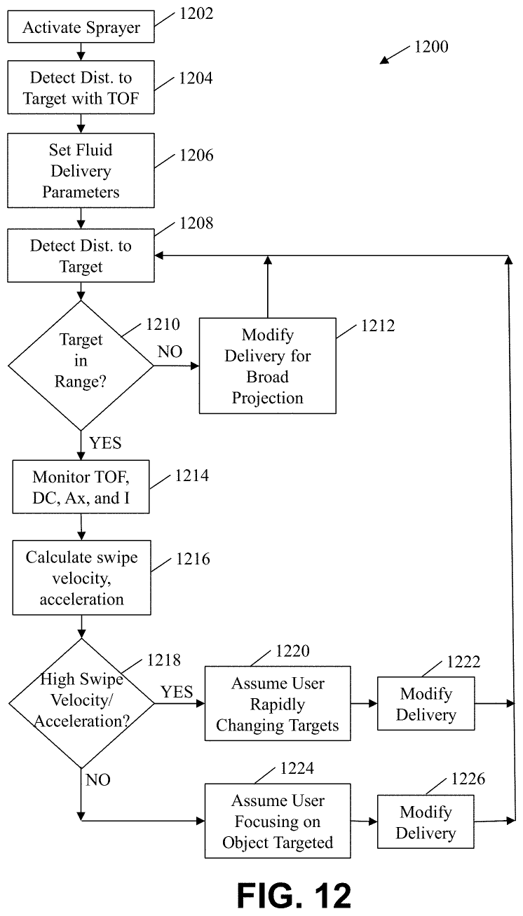

FIG. 12 is a logic diagram or methodology 1200 for an exemplary embodiment of sanitizer/disinfectant sprayer having feedback control from one or more sensors. The exemplary methodology begins at block 1202 when a user activates the sprayer. The sprayer may be activated by, for example, squeezing a trigger. When the user releases the trigger, or deactivates the sprayer, the methodology may stop. At block 1204 a distance to the targeted object is obtained. In some embodiments, the targeted object is directly in front of the sprayer wand. The distance to target object may be determined by a time of flight (TOF) sensor or other distance sensor. Based on the detected distance, a processor in the sprayer sets the initial fluid delivery parameters and begins applying fluid to the targeted object. The fluid delivery parameters, or fluid dispensing properties, may be, for example, fluid flow rate, droplet size, nozzle settings, pressure settings, spray patterns, or the like. At block 1208 the distance to the target object is again determined. At block 1210, a determination is made as to whether the target is in range. If the target is not in range, an assumption is made that the user's intention is for broad projection of spray with delivery falling onto surfaces below and the fluid deliver parameters are modified at block 1212 for broad projection delivery. The exemplary methodology loops back to block 1208.

If at block 1210 an in-range target is detected the methodology proceeds to block 1214. Several parameters may be monitored at block 1214, including but not limited to, one or more of: TOF using, for example, a time of flight sensor; azimuth horizontal angular position using, for example, a digital compass IC sensor; acceleration in the horizontal direction, using, for example, an accelerometer; and acceleration inclination, using, for example, an accelerometer. At block 1216, the data is evaluated and apparent swipe velocity and acceleration are calculated. In addition, in some embodiments, environmental factors may be included in the calculations. The environmental factors, may be, for example, school, office, hospital, doctors office, and the like.

At block 1218 a determination is made as to whether a high swipe velocity/acceleration was determined, or a low swipe acceleration was determined. If a high swipe acceleration is determined, an assumption is made at block 1220 that the user is rapidly changing targets. At block 1222, position forward in time is extrapolated based on velocities and accelerations and the fluid delivery parameters are modified at block 1222 to spray fluid with fluid delivery parameters desired for rapidly changing targets and the methodology loops back to block 1208.

If at block 1218 a determination is made that a low swipe velocity/acceleration was determined, an assumption is made that the user is focusing on the targeted object. At block 1224, position forward in time is extrapolated based on velocities and accelerations and the fluid delivery parameters are modified at block 1226 to spray fluid with fluid delivery parameters desired for directed targets and the methodology loops back to block 1208.

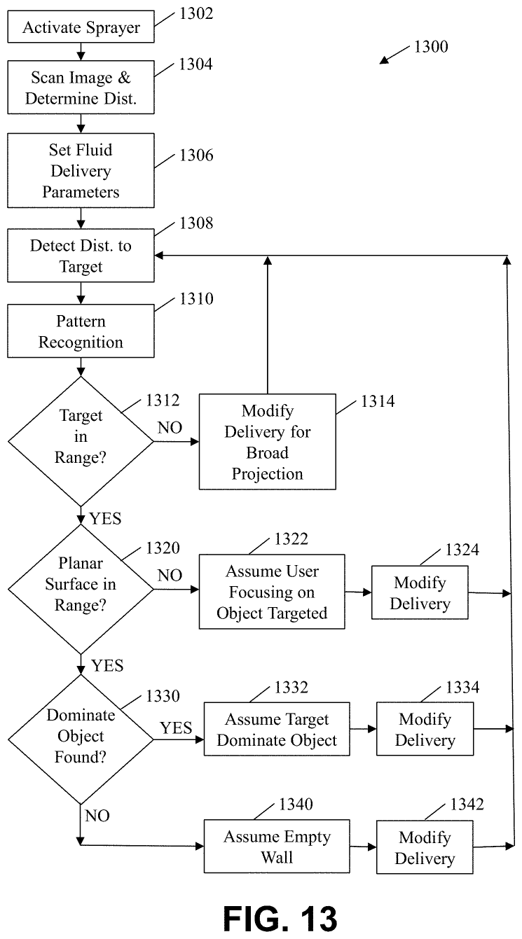

FIG. 13 is a logic diagram or methodology 1300 for an exemplary embodiment of sanitizer/disinfectant sprayer having feedback control from a Lidar sensor. The exemplary methodology begins at block 1302 when a user activates the sprayer. The sprayer may be activated by, for example, squeezing a trigger. When the user releases the trigger, or deactivates the sprayer, the methodology may stop. At block 1304, one or more image scans are med and a distance to the target is determined. At block 1306, initial fluid delivery parameters are set and the sprayer begins spraying fluid. At block 1308 a distance to the targeted object is determined. At block 1310 pattern recognition searches are conducted for planar surfaces in the field of view, e.g. wall, equipment's, office furniture. In some embodiments, the environment is also used in the calculations, such as, for example, a school, a hospital, a doctor's office, a restaurant and the like. For example, the algorithm performing the pattern recognition may search a data base of patterns for a particular environment, such as, for example, in a school setting, the data base may contain a number of different desk profiles that are common in school settings.

At block 1312, a determination is made as to whether an in-range target, such as a wall or other dominate object, is detected. If no in range planar wall or dominate object is detected, an assumption is made at block 1314 the user intends for broad projection with delivery falling onto surfaces, and the fluid delivery parameters are modified at block 1314 accordingly and the sprayer dispenses fluid using those set parameters. The exemplary methodology flows back to block 1308.

If at block 1312, a determination is made that an in-range target was detected, a determination is made at block 1320 of whether the surface is a planar surface. If the surface in range and not planer surface (not e.g. a wall, wall hanging window edge, or the like) an assumption is made at block 1322 that the user intends to deposit the sanitizer/disinfectant on the targeted object. At block 1322, one or more calculations are made. The one or more calculations may be a function of one or more of apparent swipe velocity, acceleration, distance change rate over time and accelerations. One or more of these calculations may be used to extrapolate positions forward in time. The fluid delivery parameters are modified at block 1324 to spray fluid with fluid delivery parameters in line with the one or more calculations or extrapolated positions forward in time to apply sanitizer/disinfectant to the directed targets and the methodology loops back to block 1308.

If at block 1320 a planar surface is in range, a determination is made at block 1330 to determine whether a dominate object is found in the field of view. Examples of dominate objects may be, for example, wall hanging, window edge, chair, etc. If a dominate object is found, an assumption is made that the target is the dominate object. At block 1332, one or more calculations are made. The one or more calculations may be a function of one or more of apparent swipe velocity, acceleration, distance change rate over time and accelerations. One or more of these calculations may be used to extrapolate positions forward in time. The fluid delivery parameters are modified at block 1334 to spray fluid with fluid delivery parameters in line with the one or more calculations or extrapolated positions forward in time to apply sanitizer/disinfectant to the dominate object and the methodology loops back to block 1308.

If at block 1330 a determination is made that no dominate object is found in the field of view an assumption is made that the target is an empty or flat wall. At block 1340, one or more calculations are made. The one or more calculations may be a function of one or more of apparent swipe velocity, acceleration, distance change rate over time and accelerations. One or more of these calculations may be used to extrapolate positions forward in time. The fluid delivery parameters are modified at block 1342 to spray fluid with fluid delivery parameters in line with the one or more calculations or extrapolated positions forward in time to apply sanitizer/disinfectant to the planar surface and the methodology loops back to block 1308.

The term hand-held and portable sprayer is meant to include portable sprayers that are carried around by a person during used. As such, sprayers, such as, for example, a backpack sprayer, considered to fall within the term hand-held portable sprayer.

While various inventive aspects, concepts and features of the inventions may be described and illustrated herein as embodied in combination in the exemplary embodiments, these various aspects, concepts and features may be used in many alternative embodiments, either individually or in various combinations and sub-combinations thereof. It is not the intention of the applicant to restrict or in any way limit the scope of the appended claims to such detail. Unless expressly excluded herein, all such combinations and sub-combinations are intended to be within the scope of the present inventions. Still further, while various alternative embodiments as to the various aspects, concepts and features of the inventions—such as alternative materials, structures, configurations, methods, circuits, devices and components, software, hardware, control logic, alternatives as to form, fit and function, and so on—may be described herein, such descriptions are not intended to be a complete or exhaustive list of available alternative embodiments, whether presently known or later developed. Those skilled in the art may readily adopt one or more of the inventive aspects, concepts or features into additional embodiments and uses within the scope of the present inventions even if such embodiments are not expressly disclosed herein. Additionally, even though some features, concepts or aspects of the inventions may be described herein as being a preferred arrangement or method, such description is not intended to suggest that such feature is required or necessary unless expressly so stated. Still further, exemplary or representative values and ranges may be included to assist in understanding the present disclosure; however, such values and ranges are not to be construed in a limiting sense and are intended to be critical values or ranges only if so expressly stated. Moreover, while various aspects, features and concepts may be expressly identified herein as being inventive or forming part of an invention, such identification is not intended to be exclusive, but rather there may be inventive aspects, concepts and features that are fully described herein without being expressly identified as such or as part of a specific invention. Descriptions of exemplary methods or processes are not limited to inclusion of all steps as being required in all cases, nor is the order in which the steps are presented to be construed as required or necessary unless expressly so stated.

I/We claim:

1. A hand-held portable self-adjusting sanitizer or disinfectant sprayer comprising:
a tank for holding sanitizer or disinfectant;
one or more batteries;
a motor;
a motor controller;
a pump;
the pump having a pump inlet in fluid communication with an interior of the tank;
the pump having a pump outlet;
a processor;
memory;
a dispensing wand;
one or more feedback sensors; and
logic stored on the memory;
wherein the logic stored on the memory causes the processor to change a fluid dispensing property as a function of data received from the one or more feedback sensors.

2. The hand-held portable self-adjusting sanitizer or disinfectant sprayer of claim 1 wherein the one or more feedback sensors comprises a flow sensor.

3. The hand-held portable self-adjusting sanitizer or disinfectant sprayer of claim 1 wherein the one or more feedback sensors comprises a distance sensor.

4. The hand-held portable self-adjusting sanitizer or disinfectant sprayer of claim 1 wherein the one or more feedback sensors comprises a time-of-flight sensor.

5. The hand-held portable self-adjusting sanitizer or disinfectant sprayer of claim 1 wherein the one or more feedback sensors comprises an image sensor.

6. (canceled)

7. (canceled)

8. (canceled)

9. (canceled)

10. The hand-held portable self-adjusting sanitizer or disinfectant sprayer of claim 1 further comprising logic stored on the memory for preventing operation of the self-adjusting sprayer if the battery voltage falls below a selected voltage.

11. (canceled)

12. The hand-held portable self-adjusting sanitizer or disinfectant sprayer of claim 1 further comprising logic stored on the memory for stopping the fluid flow out of the dispensing wand if the dispensing wand passes the edge of a surface.

13. (canceled)

14. (canceled)

15. (canceled)

16. The hand-held portable self-adjusting sanitizer or disinfectant sprayer of claim 1 further comprising logic stored on the memory for calculating apparent swipe velocity.

17. (canceled)

18. The hand-held portable self-adjusting sanitizer or disinfectant sprayer of claim 1 further comprising logic stored on the memory adjusting a fluid delivery rate to a first delivery rate for dispensing sanitizer/disinfectant over a broad projection and adjusting the fluid rate to a second fluid rate for dispensing sanitizer/disinfectant on a targeted object.

19. The hand-held portable self-adjusting sanitizer or disinfectant sprayer of claim 1 further comprising one or more indicators providing feedback to a user.

20. (canceled)

21. The hand-held portable self-adjusting sanitizer or disinfectant sprayer of claim 1 wherein the one or more sensors comprise a voltage sensor and if the voltage drops below a selected threshold, the processor prevents operation of the self-adjusting sprayer.

22. (canceled)

23. (canceled)

24. (canceled)

25. (canceled)

26. The hand-held portable self-adjusting sanitizer or disinfectant sprayer of claim 1 wherein the sensor senses a parameter indicative of the capacity of the battery and if the capacity of the battery drops below a selected threshold, the processor prevents operation of the self-adjusting sprayer.

27. (canceled)

28. (canceled)

29. (canceled)

30. (canceled)

31. (canceled)

32. The hand-held portable self-adjusting sanitizer or disinfectant sprayer of claim 1 wherein the fluid dispensing property is one of a fluid flow rate, a droplet size, a nozzle setting, a pressure settings, and a spray pattern.

33. A hand-held portable sanitizer/disinfectant sprayer comprising:
a tank for holding sanitizer or disinfectant;
one or more batteries;
a motor;
a motor controller;
a pump;
the pump having a pump inlet in fluid communication with an interior of the tank;
the pump having a pump outlet;
a processor;
a dispensing wand;
the dispensing wand having a dispensing wand inlet in fluid communication with the pump outlet;
and
a flow sensor;
wherein the flow sensor is in circuit communication with the processor;
wherein the processor is in circuit communication with the motor controller; and
wherein the processor provides input to the motor controller for controlling the speed of the motor that drives the pump as a function of a signal indicative of the flow rate received from the flow sensor.

34. The hand-held portable sanitizer/disinfectant sprayer of claim 33 wherein the flow sensor is an in-line flow sensor.

35. (canceled)

36. (canceled)

37. The hand-held portable sanitizer/disinfectant sprayer of claim 33 further comprising circuitry for stopping the dispenser when the voltage reaches 2 volts.

38. (canceled)

39. (canceled)

40. A hand-held portable sanitizer/disinfectant sprayer comprising:
a tank for holding sanitizer or disinfectant;
one or more batteries;
a motor;
a motor controller;
a pump;
the pump having a pump inlet in fluid communication with an interior of the tank;
the pump having a pump outlet;
a processor;
a dispensing wand;
the dispensing wand having a dispensing wand inlet in fluid communication with the pump outlet;
and
a distance sensor;
wherein the distance sensor is in circuit communication with the processor;
wherein the processor is in circuit communication with the motor controller; and
wherein the processor provides input to the motor controller for controlling the speed of the motor that drives the pump as a function of a signal indicative of the distance received from the distance sensor.

41. The hand-held portable sanitizer/disinfectant sprayer of claim 40 wherein the distance sensor is located on the dispensing wand.

42. (canceled)

43. The hand-held portable sanitizer/disinfectant sprayer of claim 40 further comprising a flow sensor located in the dispensing wand.

44. (canceled)

45. The hand-held portable sanitizer/disinfectant sprayer of claim 40 further comprising circuitry for preventing the hand-held portable sanitizer/disinfectant sprayer from operating when the battery voltage reaches 2 volts.

46. (canceled)

47. (canceled)

48. (canceled)

49. (canceled)

50. (canceled)

51. (canceled)

52. (canceled)

53. (canceled)

54. (canceled)

55. (canceled)
